(19) United States
(12) Patent Application Publication
(10) Pub. No.: US 2006/0271014 A1
(43) Pub. Date: Nov. 30, 2006

(54) Heat retention device for a syringe and methods of use

(75) Inventors: Michael R. Hynes, Chesterfield, MO (US); Frank M. Fago, Mason, OH (US)

Correspondence Address: WOOD, HERRON & EVANS, LLP, 2700 CAREW TOWER, 441 VINE STREET, CINCINNATI, OH, 45202, US

(73) Assignee: Mallinckrodt Inc.

(21) Appl. No.: 11/141,230

(22) Filed: May 31, 2005

(51) Int. Cl. A61M 31/00; B65D 83/10

(52) U.S. Cl. 604/507; 206/364

(57) ABSTRACT

A heat-retaining syringe jacket for reducing the cooling rate of a medical fluid held inside a syringe and methods of using such syringe jackets while operating a medical fluid injector. The syringe jacket includes a jacket body placed about the exterior of the syringe barrel. The jacket body may include a material that experiences a phase transition at a phase transition temperature. Additionally or alternatively, the jacket body may include a material having a high specific heat.

### FIELD OF THE INVENTION

The present invention generally relates to medical syringes and, more particularly, to medical syringes used to hold a heated medical fluid for injection into living organisms.

### BACKGROUND OF THE INVENTION

During many medical procedures, various pharmaceuticals and medical fluids are injected into living organisms for purposes of diagnosis or treatment. Commonly, injected medical fluids include, but are not limited to, x-ray contrast media or agents, flushing solutions, and other media for purposes such as enhancing diagnostic imaging in humans. Specific examples of such medical fluids are contrast media used to enhance computed tomography, magnetic resonance imaging, and angiography. The injectors used in these procedures are often automated devices that expel the fluid from a syringe, through a lumen of a tube, and into the subject. Medical fluid injectors suitable for these applications generally include relatively large volume syringes and are capable of producing relatively large flow rates and injection pressures.

Conventionally, the syringe and medical fluid therein may be warmed to a temperature near body temperature before the fluid is injected into a patient's circulatory system. Heating the medical fluid provides a benefit of reducing patient discomfort by reducing temperature mismatch. An additional benefit of heating the fluid is reduction in viscosity, which permits the medical fluid to be injected with less effort or at a higher rate.

Conventionally, the syringe carrying the medical fluid is held at approximately body temperature in a heated enclosure, such as a warmer box, and is transferred from the warmer box to the injector shortly before the medical procedure is scheduled to commence. After being removed from the warmer box, the syringe and medical fluid immediately begin to cool toward room temperature by heat loss to the surrounding environment. The extent of the cooling depends upon the time delay before the injection commences and the duration of the injection. In some instances, the medical fluid is not injected for several minutes after the syringe is removed from the warmer box. This permits the temperature of the medical fluid to drop significantly before delivering the fluid to the subject.

Conventional electrically-powered warmer blankets are used in certain medical procedures, such as computed tomography and angiography, to heat the syringe for maintaining the elevated temperature of the medical fluid. However, electrical devices like warmer blankets may unfortunately interfere with the equipment used in other types of procedures. For example, electrical current flowing through wiring of such electrically-powered warmer blankets may radiate an extraneous magnetic field. This extraneous magnetic field tends to interact with the primary magnetic field used in magnetic resonance imaging. Hence, it is generally preferred that electrically-powered warmer blankets not be used to maintain the syringe and fluid temperature in conjunction with these types of procedures.

For these and other reasons, it is desirable to provide a device that can passively maintain a medical fluid at, or near, body temperature in a syringe after the syringe and medical fluid are heated.

### SUMMARY OF INVENTION

In accordance with the principles of one aspect of the present invention, a heat retaining syringe jacket includes a jacket body with opposite first and second ends, a tubular sidewall extending between the first and second ends, and an interior cavity bounded by the tubular sidewall. The syringe jacket may further include a flange proximate the first end and extending outwardly from the tubular sidewall. During use, the syringe contains a heated medical fluid. The interior cavity is adapted to be disposed about at least a portion of the barrel of the syringe and heat exchange is promoted between the syringe jacket and the heated medical fluid.

The jacket body may include a phase change material having a phase transition temperature approximately equal to a use temperature of the heated medical fluid. The phase change material is preferably, but not necessarily, nonmagnetic. In certain embodiments of the present invention, the phase transition temperature of the phase change material is between about 80° F. and about 100° F. In other embodiments of the present invention, the phase transition temperature of the phase change material is approximately equal to a body temperature of a living organism receiving the heated medical fluid.

The jacket body may include a material having a high specific heat. For instance, the material of the jacket body may exhibit a specific heat greater than or equal to about 0.58 Btu/(lb−° F.). Suitable materials for the construction of the jacket body may include copper, aluminum, 300 series stainless steels, brass, bronze, alloys and combinations of these materials, and other materials with a suitable specific heat.

The jacket body may include a first material having a phase transition temperature between about 70° F. and about 110° F. and a second material that is substantially non-magnetic. The second material may be disposed about, and confine, the first material. The first material may be, for example, a wax-like material. The syringe jacket is preferably designed to be disposed about a majority of the syringe barrel.

Another aspect of the invention is directed to a method of using a syringe containing a medical fluid. In particular, at least a portion of the syringe is placed in a syringe jacket including a phase change material having a phase transition temperature. The syringe, the medical fluid therein, the syringe jacket, and the phase change material may be heated to an initial temperature greater than or equal to the phase transition temperature. The syringe, medical fluid, and syringe jacket are transferred to a medical fluid injector. The medical fluid injector is operated to inject an amount of the medical fluid into a patient. While operating the medical fluid injector to inject the amount of medical fluid, the temperature of the medical fluid is maintained approximately at the phase transition temperature due, in large part, to the design of the syringe jacket.

Yet another aspect of the invention is directed to a method of using a syringe containing a medical fluid. In particular, at least a portion of the syringe is placed in a syringe jacket that is effective to increase a thermal mass of the syringe and the medical fluid. The syringe, medical fluid, and syringe jacket may be heated to an initial temperature greater than or equal to a use temperature of the medical fluid for injection into a patient. The syringe, medical fluid, and syringe jacket are transferred to a medical fluid injector, which is operated to inject an amount of the medical fluid into a patient. While operating the medical fluid injector to inject the amount, the heat loss of the medical fluid is reduced due, in large part, to the design of the syringe jacket.

A syringe jacket, or other heat retention device of the invention, may be used to maintain the temperature of any medical fluid, such as an injectable pharmaceutical, capable of being held by a syringe. Beneficially, syringe jackets and heat retention devices of the invention are useable in environments in which conventional electrical devices, such as warmer blankets, are not desired, easily tolerated, or permitted. For example, the heat retention device of the present invention may be used to maintain the temperature of contrast media used in magnetic resonance (MR) applications because the syringe and associated syringe jacket may be held in close proximity to a high field strength magnetic field without disrupting or perturbing the field lines of the magnetic field.

These and other advantages of the present invention shall become more apparent from the accompanying drawings and description thereof.

### BRIEF DESCRIPTION OF DRAWINGS

The accompanying drawings, which are incorporated in and constitute a part of this specification, illustrate embodiments of the invention and, together with a general description of the invention given above, and the detailed description given below, serve to explain the principles of the invention.

FIG. 1 is a perspective view showing a heat retaining syringe jacket for use with a syringe;

FIG. 2 is a top view of the assembled heat retaining syringe jacket and syringe of FIG. 1;

FIG. 3 is a top view similar to FIG. 2, but partially broken away, of a heat retaining syringe jacket constructed in accordance with an alternative embodiment of the present invention;

FIG. 4 is a graphical representation of cooling profiles for various embodiments of the heat retaining syringe jacket of the present invention and a cooling profile for a prior art syringe lacking the heat retaining syringe jacket;

FIG. 5 is a perspective view of an injector holding two syringes each coupled with a heat retaining syringe jacket of the present invention; and

FIG. 6 is a partial cross-sectional view taken generally along line 6-6 in FIG. 5 with the syringe elevated above the syringe cradle for clarity.

### DETAILED DESCRIPTION

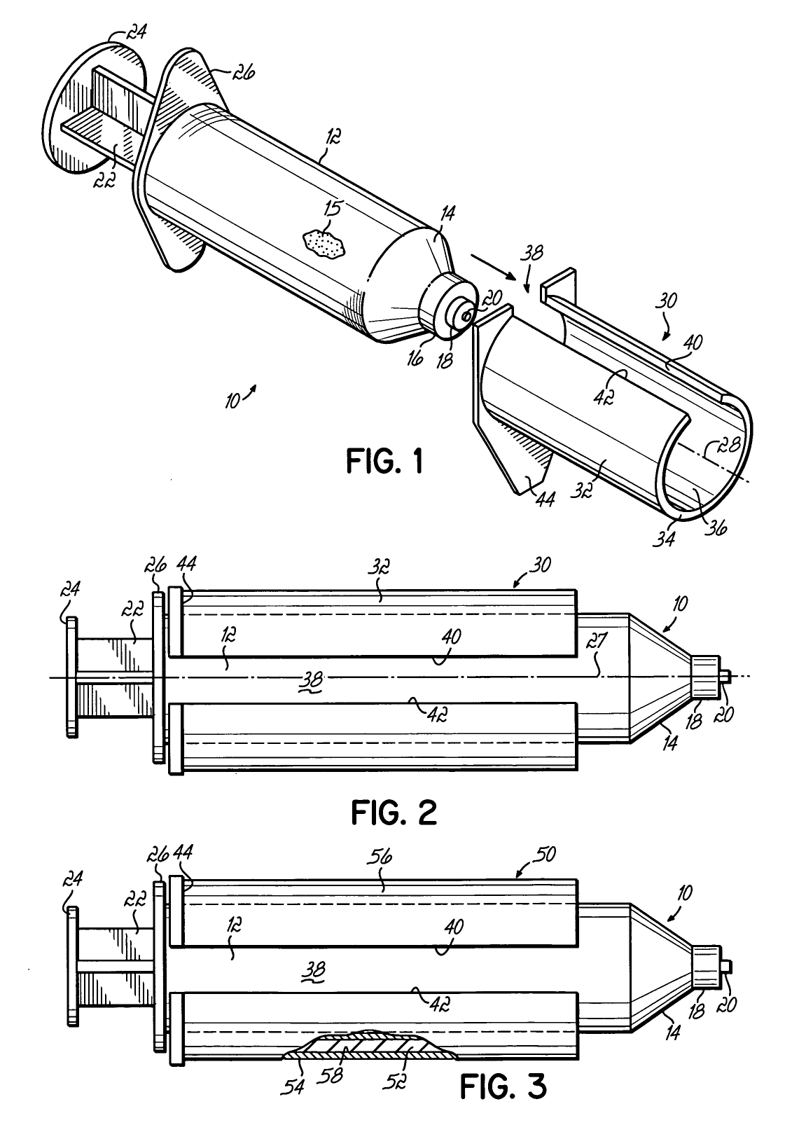

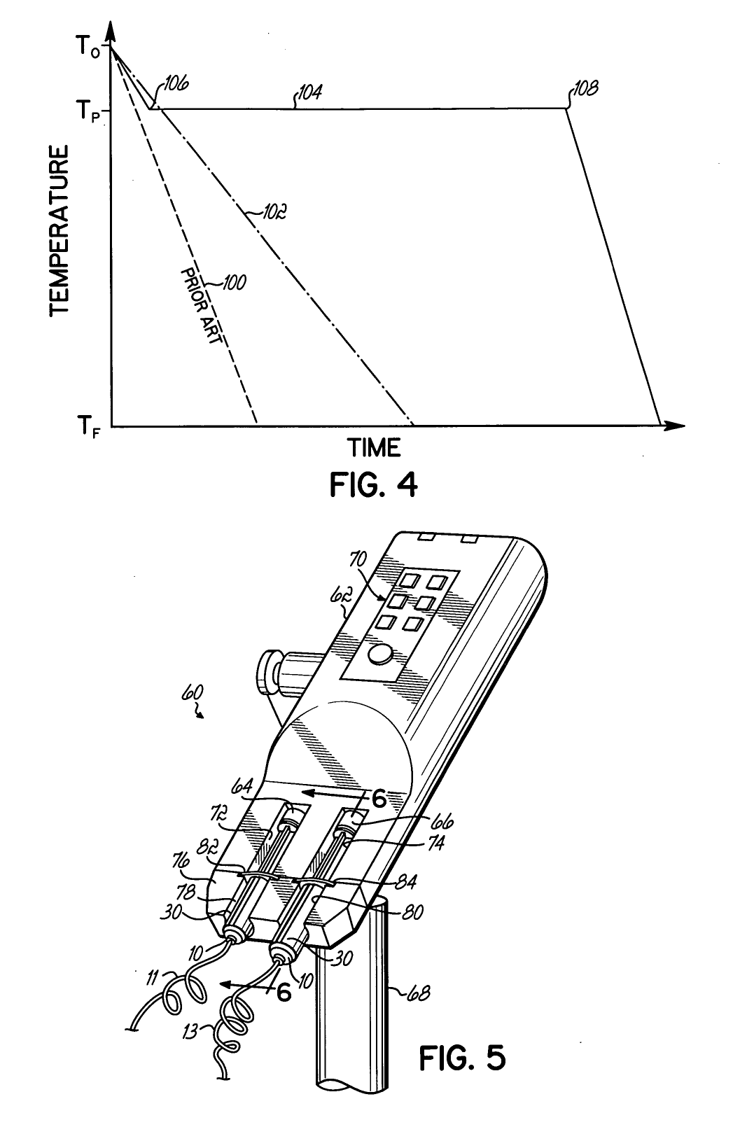

With reference to FIGS. 1 and 2, a syringe 10 generally includes a tubular sidewall 12 that may be in the form of an exterior cylindrical and hollow barrel. The forward end of syringe sidewall 12 is integral with a tapered front wall section 14 generally of frustoconical shape. A neck 16, terminating in a discharge tip 18, generally extends forwardly from and may be integral with the front wall section 14. The discharge tip 18 of the syringe 10 generally contains an outlet 20 coupled in fluid communication with an internal syringe cavity or reservoir defined by the collective space bounding inside the neck 18, the front wall section 14, and the syringe sidewall 12. Tubing 11 (FIG. 4) may be attached to the discharge tip 18 by any of a number of appropriate manners. For example, the tubing 11 may be attached to the discharge tip 18 by a conventional needle or cannula fitting (not shown), such as a luer, Luer-Lok, etc., that couples the outlet 20 in communication with the lumen of a length of the tubing 11 and defines a fluid delivery path to the subject.

The open rearward end of the syringe sidewall 12 receives a syringe plunger 22 with a forward facing head (not shown) preferably, but not necessarily, contoured to at least generally conform to the shape of the interior of the front wall section 14. The head of the syringe plunger 22 is preferably snugly slidable within the syringe sidewall 12 and generally has a forward facing surface that tends to contact a medical fluid 15 inside the reservoir. The reservoir of the syringe 10 may be said to have a variable volume contingent upon the position of the head of syringe plunger 22 relative to the front wall section 14. As the syringe plunger 22 is advanced toward the front wall section 14 by applying a force to an exposed rearward end 24, medical fluid 15 held inside the reservoir is ejected from the outlet 20. The syringe plunger 22 preferably has a substantially sealed engagement with the interior of the syringe sidewall 12 so that little or no medical fluid 15 inside the reservoir escapes rearwardly past the syringe plunger 22 as the syringe plunger 22 is advanced relative to the syringe sidewall 12.

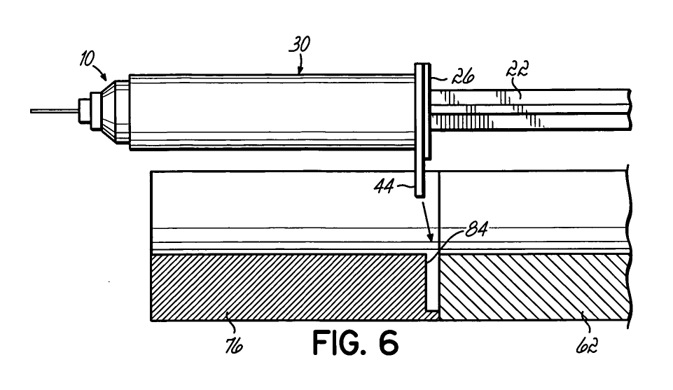

The syringe 10 includes a mating section 26, which may be in the form of a radially outwardly extending flange. The syringe mating section 26 is oriented in a plane orthogonal to a longitudinal axis 27 of the syringe 10 and extends along the length of the syringe 10. The mating section 26, which is integral with a rearward end of the syringe sidewall 12, facilitates the connection of the syringe 10 to an injector 60 (FIG. 5).

With continued reference to FIGS. 1 and 2 and in accordance with the principles of the invention, a syringe jacket 30 includes a jacket body 32 with a substantially tubular jacket sidewall 34 defining a cavity 36 shaped and sized to receive at least a portion of the syringe sidewall 12. The jacket body 32 may be substantially solid, substantially hollow, have one or more cavities defined therein, or surround an interior material in the jacket body 32. The jacket sidewall 34 extends along and is centered about a longitudinal axis 28. The cross-sectional profile of the sidewall 34 viewed in a direction parallel to longitudinal axis 28 is generally C-shaped with a curved or arcuate and tube-like section and a gap in the tube-like section defined by a lengthwise-extending slot 38.

When the syringe jacket 30 is fitted about the syringe 10, the jacket sidewall 34 is in a relationship with the syringe 10, preferably either in contact or in a confronting relationship, that promotes heat exchange with at least a portion of the syringe sidewall 12. In addition, the longitudinal axis 28 is substantially aligned with the longitudinal axis 27 of the syringe 10. Preferably, the syringe jacket 30 is adapted to be disposed about a majority of the sidewall 12. The arrangement of the jacket body 32 and syringe 10 promotes heat transfer between the jacket sidewall 34 and the syringe sidewall 12 and, subsequently, through the syringe sidewall 12 to the medical fluid 15 held inside the reservoir of the syringe 10. The arrangement also situates the jacket body 32 as a thermally-insulating barrier that prevents or, at the least, reduces heat loss from the covered portion of the syringe sidewall 12 to the surrounding environment of the syringe 10 and jacket body 32.

Slot 38, which extends along the axial length of the jacket body 32, separates a pair of parallel edges 40, 42 of the jacket sidewall 34. The slot 38 permits an observer to perceive the position of the plunger 22 and its head, for example, during a procedure. The presence of the slot 38 also provides access to cavity 36, which permits the syringe 10 to remain coupled with the tubing during loading and unloading. While it is generally preferred that the syringe jacket 30 include the slot 38, some embodiments of the invention may include a syringe jacket that is completely devoid of the slot 38, that has one or more slots extending only partially along the length of the syringe jacket 30, that has one or more apertures extending through the syringe jacket 30, and/or that has one or more slots that is not in alignment with the reference axis 28.

The syringe jacket 30 includes a mating section 44, which may be in the form of a radially outwardly extending flange, integrally formed with a rearward end of the jacket sidewall 34. The mating section 44 cooperates with the mating section 26 of the syringe 10 to facilitate the connection of the syringe 10 and syringe jacket 30 to an injector 60 (FIG. 5). The mating section 44 is oriented in a plane orthogonal to the longitudinal axis 28. When the syringe jacket 30 is fitted about the syringe 10, the mating section 44 of the syringe jacket 30 is preferably positioned proximate to the mating section 26 of syringe 10 and is preferably oriented to at least generally align with a portion of mating section 26.

The syringe jacket 30 is preferably reusable. After an injection procedure is concluded, the syringe 10 and syringe jacket 30 may be separated from one another. The syringe 10 may be discarded or reused. Preferably, the syringe jacket 30 is sterilized and cleaned and then fitted about another syringe 10 for use in a future injection procedure. The ability to reuse the syringe jacket 30 reduces the effective cost of the syringe jacket 30.

The syringe jacket 30 operates to reduce a rate at which the syringe 10 and medical fluid 15 inside the reservoir of the syringe 10 cool after being heated to an initial temperature. Typically, the syringe 10, medical fluid 15, and syringe jacket 30 are heated to the initial temperature, which preferably exceeds room or ambient temperature (i.e., about 70° F. (21° C.)). In use, the syringe 10, medical fluid 15 held by the syringe 10, and the syringe jacket 30 are placed into a heated enclosure, or the like, and warmed to an elevated target temperature of use during the injection procedure or, alternatively, to a target temperature exceeding the use temperature at which the medical fluid 15 is to be injected from the syringe 10 into a subject. As one specific example, the target temperature of use may be about 99° F. (37° C.), which is approximately equal to normal human body temperature. In this specific instance, the syringe 10, medical fluid 15 held by the syringe 10, and syringe jacket 30 are heated to the use temperature of 99° F. or above before removing the syringe 10 from the heated enclosure and commencing the injection procedure introducing the heated medical fluid 15 into the subject.

The addition of the syringe jacket 30 effectively increases the thermal mass of the syringe 10 and medical fluid 15 held inside syringe 10. The syringe 10, medical fluid 15, and syringe jacket 30 are assumed to cool approximately at the same rate because of their thermal coupling. By increasing the thermal mass, the cooling rate of the syringe 10, medical fluid 15, and syringe jacket 30 is reduced because the change in temperature during cooling is inversely proportional to the mass of the combined structure. The cooling rate of the syringe jacket 30 is also inversely proportional to the specific heat of the constituent material.

The specific heat of a substance, as used herein, represents the product of the specific heat capacity of the substance and the specific gravity of the substance. The specific gravity, which is also known as relative density, is a dimensionless measure of the density of a substance divided by the density of water. Specific heat capacity of a substance represents the amount of heat energy required to raise one (1) gram of the substance by one (1) degree Celsius.

To limit the size and weight of the syringe jacket 30, the syringe jacket 30 is preferably formed from a constituent material having or exhibiting a room temperature specific heat greater than or equal to about 0.58 BTU/(lb·° F.). As non-limiting examples of suitable materials, the material constituting the syringe jacket 30 may be copper having a specific heat of about 0.81 BTU/(lb·° F.) (i.e., 0.093 BTU/(lb·° F.)·8.7), aluminum having a specific heat of about 0.58 BTU/(lb·° F.) (i.e., 0.22 BTU/(lb·° F.)·2.6), 300 series stainless steels having a specific heat of about 0.92 BTU/(lb·° F.) (i.e., 0.12 BTU/(lb·° F.)·7.7), brass having a specific heat of about 0.76 BTU/(lb·° F.) (i.e., 0.09 BTU/(lb·° F.)·8.5), bronze having a specific heat of about 0.83 BTU/(lb·° F.) (i.e., 0.104 BTU/(lb·° F.)·8.0), and alloys or combinations of these materials. However, the invention contemplates that the syringe jacket 30 may be formed from multiple materials chosen such that the composite heat capacity is greater than or equal to the heat capacity of aluminum (i.e., about 0.58 BTU/(lb·° F.)), which admits to combinations of a material having a heat capacity less than that of aluminum with a material having a heat capacity greater than that of aluminum.

Preferably, the material forming the syringe jacket 30 is also substantially non-magnetic, which permits the syringe jacket 30 to be placed in a magnetic field environment without perturbing the field lines of the magnetic field. However, the syringe jacket 30 may also be used in environments lacking an artificial magnetic field used for diagnostic or therapeutic reasons during a medical procedure related to the medical fluid injection. The syringe jacket 30 may be reused because the syringe jacket 30 is not physically altered by the temperature changes.

With reference to FIG. 3 in which like reference numerals refer to like features in FIG. 2 and in accordance with another embodiment of the present invention, a syringe jacket 50 may incorporate an amount or quantity of a phase change material 52 that operates to maintain the temperature of the medical fluid 15 held inside syringe 10. The phase change material 52 is preferably in a liquid state at, or above, a phase transition temperature from the solid state to the liquid state and, therefore, must be contained or otherwise confined in some manner to prevent leakage of the liquid from the syringe jacket 50. Accordingly, a jacket sidewall 54 of a jacket body 56 includes a closed or sealed hollow chamber or compartment 58 filled by and encapsulating the quantity of the phase change material 52. In a liquid or gaseous state, the phase change material 52 is preferably contained within the space provided inside the compartment 58 and, consequently, is not able to migrate or leak outside of the syringe jacket 50 or otherwise escape from the syringe jacket 50.

The phase change material 52 in compartment 58, when changing phase exothermically (e.g., from a liquid to a solid), preferably maintains a substantially constant phase transition temperature even though heat energy is being removed or dissipated because the exothermic phase transition continuously releases heat energy while at the phase transition point between phases (e.g., the melting point associated with phase change from a solid to a liquid). Any heat lost by the syringe 10 and enclosed medical fluid 15 is generally replaced by the heat of fusion released by the phase change material 52 as the phase change material 52 transitions (e.g., from the liquid phase to the solid phase).

The syringe 10 and the medical fluid 15 held inside the syringe 10 will cool to the temperature of the phase change material 52 and remain at the transition temperature irregardless of the initial temperature of the syringe 10, medical fluid 15, and phase change material 52. That is because the initial and final phases of the phase change material 52 are in equilibrium at the transition temperature until all the initial phase is converted to the final phase. As heat is removed from the phase change material 52 by cooling and transfer to the syringe 10, the medical fluid 15, and the environment surrounding the syringe jacket 50, the lost heat is replaced by converting some initial phase of the phase change material 52 into final phase. Thus, the temperature of the syringe 10 and medical fluid 15 may be controlled and maintained at a use temperature by surrounding the syringe 10 and medical fluid 15 with the syringe jacket 50 including the phase change material 52. This effectively reduces the cooling rate of the medical fluid 15, which may operate to lengthen the time at which the medical fluid 15 is at or near the target temperature during the injection procedure.

The phase change material 52 may be any suitable organic or inorganic substance selected to have a phase transition temperature such that the phase change material 52 will undergo a phase transition at or near the target use temperature of the medical fluid 15 for a given procedure. The phase transition temperature of phase change material 52 may be above the use temperature, approximately equal to the use temperature, or less than the use temperature. For example, the phase change material 52 may be the inorganic elemental substance gallium, which has or exhibits a phase transition temperature or melting point of about 30° C. (85.6° F.), an inorganic compound like iron (III) chloride, hexahydrate, which has a melting point of about 37° C. (about 99° F.), or any organic wax-like material having a melting point at, or near, the target use temperature. The compositions of such wax-like materials are understood by persons of ordinary skill in the art. As other examples, the phase change material 52 may be selected from inorganic substances, such as heneicosane (C₂₁H₄₄), which has a melting point of about 39° C. (about 102° F.), eicosane C₂₀H₄₂, which has a melting point of about 37° C. (about 99° F.), nonadecane C₁₉H₄₀ which has a melting point of about 33° C. (about 91° F.), and beta theobroma oil or cocoa butter that has a melting point of about 33° C. A person of ordinary skill in the art will readily appreciate other organic and inorganic substances with suitable phase transition temperatures for use as the phase change material 52.

It is sometimes preferably that the phase transition temperature of the phase change material 52 is approximately equal to a body temperature of a living organism receiving the medical fluid. As such, for injections into humans, the phase change material 52 may have a phase transition temperature (i.e., exhibit a phase change) between about 70° F. and about 110° F. (i.e., near human body temperature). Alternatively, the phase transition temperature of phase change material 52 may be between about 80° F. and about 100° F. Alternatively, the phase transition temperature of phase change material 52 may be between about 80° F. and about 100° F. Alternatively, the phase transition temperature of phase change material 52 may be between about 85° F. and about 100° F. Alternatively, the phase transition temperature of phase change material 52 may be between about 90° F. and about 100° F. Alternatively, the phase transition temperature of phase change material 52 may be about 90° F. As understood by persons of ordinary skill in the art, the phase change exhibited by phase change material 52 during cooling may be from a liquid to a solid or, alternatively, from a gas or vapor to a liquid.

Preferably, the material forming the syringe jacket 50 and the phase change material 52 are both substantially non-magnetic or, at the least, the material forming the syringe jacket 50 is substantially non-magentic, which promotes use of the syringe jacket 50 in a magnetic field environment without perturbing the field lines of the magnetic field, although the invention is not so limited as this compatibility is not required in environments lacking a magnetic field. The selected phase change material 52 confined inside compartment 58 and, therefore, wetting portions of the jacket sidewall 54 is preferably chemically compatible and substantially stable with the material constituting the jacket sidewall 54. The material forming the syringe jacket 50 may be any of the materials listed herein that are characterized by, or exhibit, a specific heat greater than or equal to about 0.58 Btu/(lb−° F.). However, materials having a lower specific heat may be used because of the presence of the phase change material 52. The syringe jacket 50 may be reused because the phase transition experienced by phase change material 52 is reversible.

With reference to FIG. 4, a schematic representation is shown of a theoretical prior art cooling profile of the heated medical fluid 15 held by a conventional heated syringe 10, a theoretical cooling profile of the heated medical fluid 15 held by a conventional heated syringe 10 used in combination with jacket 30 of the present invention, and a theoretical cooling profile of the heated medical fluid 15 held by a conventional heated syringe 10 used in combination with jacket 50 of the present invention. The fluid temperature is the abscissa, and the ordinate is the cumulative time originating at an initial time at which the syringe 10 and syringe jacket 30, 50 are removed from a heated enclosure (not shown). The heated syringe 10, medical fluid 15, and jacket 30, 50 are at an initial temperature when removed from the heated enclosure.

Line 100 in FIG. 4 illustrates the representative cooling profile for a conventional syringe, similar to syringe 10, lacking a syringe jacket holding a heated medical fluid after removal from the heated enclosure. The fluid temperature cools at a linear rate from an initial temperature, T₀, to a final temperature T_{F}, that is less than the initial temperature and that may be an ambient temperature. A use temperature desired for the injection procedure is typically defined between the initial and final temperatures, but may equal the initial temperature or be greater than the initial temperature.

Line 102 in FIG. 4 illustrates the representative cooling profile for the heated medical fluid 15 held by syringe 10 when a syringe jacket 30 is disposed thereabout and after removal from the heated enclosure. As is apparent from line 102, the fluid temperature cools at a linear rate from the initial temperature with a smaller slope than line 100. This implies that the heated medical fluid 15 will, on average, be injected at a temperature closer to the use temperature because of the presence of syringe jacket 30. The reduction in the cooling rate of the medical fluid 15 results in large part from the thermal mass and thermal insulation introduced by the presence of the syringe jacket 30.

Line 104 in FIG. 4 illustrates the representative cooling profile for the medical fluid 15 held by the syringe 10 when the syringe jacket 50 is disposed thereabout and after removal from the heated enclosure. When the syringe 10, medical fluid 15, and syringe jacket 50 are removed from the heated enclosure, the heated syringe 10, medical fluid 15, and syringe jacket 50 including phase change material 52 initially cool down from the initial temperature in the usual way as heat is lost in proportion to their individual heat capacities. When the cooling phase change material 52 reaches the phase transition temperature (T_{P}) at point 106, some of the initial phase of the phase change material 52 begins to convert to the final phase. As more heat is removed, more of the initial phase of the phase change material 52 converts to the final phase. Because this phase transition releases heat energy, the temperature of the phase change material 52 stays substantially constant. That is, the heat that the heated medical fluid 15 loses by cooling is replenished by heat originating from conversion of the initial phase of the heat change material 52 to the final phase.

While both the initial and final phases are in equilibrium, the temperature of the phase change material 52 remains at the transition (i.e., fusion) point. Heat is continuously transferred from the phase change material 52 to the syringe 10 and medical fluid 15, which replaces heat lost by the syringe 10 and medical fluid 15 to the surrounding environment. Fluid heat loss is also reduced across regions of the syringe 10 separated and thermally insulated from the surrounding environment by the syringe jacket 50. As soon as all the initial phase has been converted to the final phase at point 108, the temperature of the phase change material 52 will begin to drop again as the final phase cools passively in the usual way toward the final temperature. The heated syringe 10 and medical fluid 15 will also cool toward the final temperature. The added thermal mass of the syringe jacket 50 will also reduce the cooling rate as the heated syringe 10 and medical fluid 15 further cool. Preferably, the injection procedure concludes either before or shortly after the phase transition of the phase change material 52 is complete so that the medical fluid 15 is maintained at or near the phase transition temperature during injection. [0046 1 With reference to FIGS. 5 and 6 in which like reference numerals refer to like features in FIGS. 1 and 2, a pair of syringes 10 is mounted to a powerhead 62 of a medical fluid injector 60. Each of the syringes 10 includes a heat retaining syringe jacket 30 with the sidewall 34 of the jacket body 32 in a surrounding relationship with a length or portion of the syringe sidewall 12. The rearward end 24 of each syringe plunger 22, when the corresponding syringe 10 is mounted in injector 60, is located proximal to and in substantial alignment with a corresponding one of a pair of plunger drive rams 64, 66 of the injector 60. Each of the plunger drive rams 64, 66 is coupled either passively or actively with some level of positive gripping with the corresponding syringe plunger 22. Thus, each plunger drive ram 64, 66 may be advanced, at the same time advancing the syringe plunger 22 within the corresponding syringe 10.

Each of the plunger drive rams 64, 66 is driven by a motor (not shown) to move in a forward motion and, thus, the syringe plunger 22 of the corresponding syringe 10 is moved in a forward motion along its axis of symmetry 28 to inject medical fluid 15 into a human or animal subject. Heated medical fluid 15 is discharged from the corresponding outlet 20 of each of the syringes 10 through the associated tubing 11, 13 when the corresponding one of the plunger drive rams 64, 66 is advanced to move the associated syringe plunger 22. The plunger drive rams 64, 66 are also movable in a rearward direction to, for example, withdraw the drive rams 64, 66 and release the corresponding syringe 10.

The powerhead 62 of the injector 60, which is supported by a base 68, includes a user-injector 70 interface with, for example, controls to control and/or program movement of the plunger drive rams 64, 66 and a display screen that provides information regarding the injection procedure. The powerhead 62 includes a pair of elongate syringe cradles or grooves 72, 74 each capable of holding and laterally constraining one of the syringes 10 against lateral movement during the injection procedure. Each of the drive rams 64, 66 moves in a direction generally parallel to the major axis of the corresponding one of the grooves 72, 74.

The powerhead 62 of the injector 60 features a removable syringe mounting or adaptor 76 that includes a pair of grooves 78, 80 each of which, when the adaptor 76 is mounted to the powerhead 62, aligns with one of the grooves 72, 74 in the powerhead 62. Defined at the boundary between the powerhead 62 and the adapter 76, at the intersection between groove 78 in the adaptor 76 and groove 72 in the powerhead 62, is a coupling element 82 in the form of a recess that extends across the width of the coinciding grooves 72, 78. Similarly, defined at the boundary between the powerhead 62 and the adapter 76, at the intersection between the groove 80 in the adaptor 76 with the grooves 74 in the powerhead 62, is a coupling element 84 also in the form of a recess that extends across the width of the coinciding grooves 74, 80. Each of the coupling elements 82, 84 is sized and shaped to match and receive the mating section 26 of syringe 10 and the mating section 44 of the syringe jacket 30.

As the syringe 10 and syringe jacket 30 are positioned in proximity to one of the grooves 72, 74 and moved downwardly toward the base 68 of the injector 60 so as to be inserted in the corresponding one of grooves 72, 74, the mating sections 26, 44 are received in and engaged by the corresponding one of the coupling elements 82, 84. The engagement between the coupling elements 82, 84 and the mating sections 26, 44 holds the syringe 10 and syringe jacket 30 stationary when the corresponding one of the drive rams 64, 66 is moved.

After transferring the heated syringe 10, the heated medical fluid 15, and the heated syringe jacket 30 to the injector 60, and while the injector 60 is operating and/or after the injector 60 operates, a diagnostic imaging procedure may be performed on the patient injected by operation of injector 60 with an amount the heated medical fluid 15. This diagnostic imaging procedure may utilize a magnetic field as part of the imaging process. The syringe jacket 30 reduces heat loss of the heated medical fluid 15 while operating the injector 60 to inject the amount of the heated medical fluid 15.

The preceding description is equally applicable to syringe jacket 50. According, the temperature of the heated medical fluid 15 may be maintained approximately at the phase transition temperature of the phase change material 52 while operating the injector 60 to inject an amount of the heated medical fluid 15. The invention contemplates that the syringe jackets 30, 50 may be used with various different injectors and that use is not limited to use with a medical fluid injector having the specific construction of injector 60.

While the present invention has been illustrated by a description of various preferred embodiments and while these embodiments have been described in considerable detail in order to describe the best mode of practicing the invention, it is not the intention of applicants to restrict or in any way limit the scope of the appended claims to such detail. Additional advantages and modifications within the spirit and scope of the invention will readily appear to those skilled in the art. The invention itself should only be defined by the appended claims, wherein we claim:

1. a heat retaining syringe jacket for use with a syringe having a barrel, the syringe confining a heated medical fluid capable of being injected into a patient at a use temperature, comprising:
a jacket body including opposite first and second ends, a tubular sidewall extending between said first and second ends, and an interior cavity bounded by said tubular sidewall, said interior cavity adapted to be disposed about at least a portion of the barrel of the syringe, and said jacket body including a phase change material having a phase transition temperature approximately equal to the use temperature.

2. The syringe jacket of claim 1 wherein said interior cavity is sized and shaped such that said tubular sidewall at least partially contacts the barrel of the syringe circumferentially when the syringe is received in said interior cavity.

3. The syringe jacket of claim 2 wherein said interior is sized and shaped such that a portion of the barrel projects from said interior cavity at said second end.

4. The syringe jacket of claim 1 wherein said jacket body includes a slot extending between said first and second ends and penetrating through said tubular sidewall to permit access to said interior cavity.

5. The syringe jacket of claim 1 wherein said tubular sidewall of said jacket body includes a compartment and said phase change material is confined within said compartment.

6. The syringe jacket of claim 1 wherein said phase change material is nonmagnetic.

7. The syringe jacket of claim 1 wherein the phase transition temperature of said phase change material is between about 70° F. and about 110° F.

8. The syringe jacket of claim 1 wherein said syringe jacket is operative to maintain the heated medical fluid in the syringe approximately at the phase transition temperature.

9. The syringe jacket of claim 1 wherein said syringe jacket is effective to reduce a cooling rate of the heated medical fluid by maintaining the heated medical fluid approximately at the phase transition temperature.

10. The syringe jacket of claim 1 wherein said phase transition temperature of said phase change material is greater than the use temperature of the heated medical fluid.

11. The syringe jacket of claim 1 wherein said phase change material experiences a phase change from a liquid to a solid at said phase transition temperature.

12. The syringe jacket of claim 1 further comprising:
a flange proximate said first end and extending outwardly from said tubular sidewall.

13. A heat retaining syringe jacket for use with a syringe having a barrel and a medical fluid confined inside the barrel, comprising:
a jacket body including opposite first and second ends, a tubular sidewall extending between said first and second ends, and an interior cavity bounded by said tubular sidewall, said interior cavity adapted to be disposed about at least a portion of the barrel of the syringe, and said jacket body including a material having a specific heat greater than or equal to about 0.58 Btu/(lb−° F).

14. The syringe jacket of claim 13 wherein said interior cavity is sized and shaped such that said tubular sidewall at least partially contacts the barrel of the syringe circumferentially when the syringe is received in said interior cavity.

15. The syringe jacket of claim 14 wherein said interior is sized and shaped such that a portion of the barrel projects from said interior cavity at said second end.

16. The syringe jacket of claim 13 wherein said jacket body includes a slot extending between said first and second ends and penetrating through said tubular sidewall to permit access to said interior cavity.

17. The syringe jacket of claim 13 wherein said material is non-magnetic.

18. The syringe jacket of claim 13 wherein said syringe jacket is effective to reduce a cooling rate of the heated medical fluid.

19. The syringe jacket of claim 13 further comprising:
a flange proximate said first end and extending outwardly from said tubular sidewall.

20. A method of using a syringe containing a medical fluid, comprising:
placing at least a portion of the syringe in a syringe jacket including a phase change material having a phase transition temperature;
transferring the syringe, the syringe jacket, and the medical fluid to a medical fluid injector;
operating the medical fluid injector to inject an amount of the medical fluid into a patient; and
maintaining the temperature of the medical fluid approximately at the phase transition temperature while operating the medical fluid injector to inject the amount of the medical fluid.

21. The method of claim 20 wherein the amount is less than a fluid capacity of the syringe.

22. The method of claim 20 further comprising:
heating the syringe, the medical fluid, the syringe jacket, and the phase change material to an initial temperature greater than or equal to the phase transition temperature.

23. The method of claim 22 wherein the initial temperature is greater than the phase transition temperature, and further comprising:
allowing the syringe, the medical fluid, and the syringe jacket to cool from the initial temperature to the phase transition temperature while operating the medical fluid injector.

24. The method of claim 20 further comprising:
allowing the phase change material to complete a phase transition at the phase transition temperature while operating the medical fluid injector; and
continuing to operate the medical fluid injector after the medical fluid cools below the phase transition temperature.

25. The method of claim 20 further comprising:
installing the syringe and the jacket in a syringe mounting of the medical fluid injector; and
holding the syringe and the jacket stationary within the syringe mounting while operating the medical fluid injector.

26. The method of claim 25 further comprising:
removing the syringe mounting from the medical injector after the medical fluid injector is operated.

27. The method of claim 20 further comprising:
performing diagnostic imaging utilizing a magnetic field after transferring the syringe, the medical fluid, and the syringe jacket to the medical fluid injector.

28. A method of using a syringe containing a medical fluid, comprising:
placing at least a portion of the syringe in a syringe jacket effective to increase a thermal mass of the syringe and the medical fluid;
transferring the syringe, the medical fluid, and the syringe jacket to a medical fluid injector;
operating the medical fluid injector to inject an amount of the medical fluid into a patient; and
reducing heat loss of the medical fluid with the syringe jacket while operating the medical fluid injector to inject the amount of the medical fluid.

29. The method of claim 28 wherein the amount is less than a fluid capacity of the syringe.

30. The method of claim 28 further comprising:
installing the syringe and the jacket in a syringe mounting of the medical fluid injector; and
holding the syringe and the jacket stationary within the syringe mounting while operating the medical fluid injector.

31. The method of claim 30 further comprising:
removing the syringe mounting from the medical injector after the medical fluid injector is operated.

32. The method of claim 28 further comprising:
heating the syringe, the medical fluid, and the syringe jacket to an initial temperature greater than or equal to a use temperature of the heated medical fluid for injection into a patient.

33. The method of claim 32 wherein reducing heat loss comprises:
maintaining the heated medical fluid at or above the use temperature while operating the medical fluid injector.

34. The method of claim 28 wherein the syringe jacket further includes a phase change material having a phase transition temperature, and reducing heat loss comprises:
maintaining the temperature of the heated medical fluid approximately at the phase transition temperature while operating the medical fluid injector.

35. The method of claim 28 further comprising:
performing diagnostic imaging utilizing a magnetic field after transferring the syringe, the medical fluid, and the syringe jacket to the medical fluid injector.

36. A syringe jacket for use with a syringe having a medical fluid disposed therein, the syringe jacket adapted to be disposed about a majority of a syringe barrel, said syringe jacket comprising:
a first material having a phase transition temperature between about 70° F. and about 110° F.; and
a second material that is substantially non-magnetic.

37. The syringe jacket of claim 36 wherein the second material exhibits a specific heat greater than or equal to about 0.58 Btu/(lb−° F.).

38. The syringe jacket of claim 36 wherein said second material is disposed about and confines said first material.

39. The syringe jacket of claim 36 wherein said first material is a wax-like material.

40. The syringe jacket of claim 36 wherein said first material has a phase transition temperature of about 90° F.

42. The syringe jacket of claim 36 wherein said first material has a phase transition temperature between about 80° F. and about 100° F.

42. The syringe jacket of claim 36 wherein said first material has a phase transition temperature between about 85° F. and about 100° F.

43. The syringe jacket of claim 36 wherein said first material has a phase transition temperature between about 90° F. and about 100° F.

44. The syringe jacket of claim 36 wherein said first material experiences a phase change from a liquid to a solid at said phase transition temperature.

45. The syringe jacket of claim 36 wherein said first material is substantially non-magnetic.
